(19) United States
(12) Patent Application Publication
(10) Pub. No.: US 2010/0198017 A1
(43) Pub. Date: Aug. 5, 2010

(54) Laryngoscope Blade

(75) Inventor: Louise D. Raspallo, Cumberland, RI (US)

Correspondence Address: FISH & RICHARDSON P.C., P.O. BOX 1022, MINNEAPOLIS, MN, 55440-1022, US

(73) Assignee: TAPS, LLC, Lincoln, RI (US)

(21) Appl. No.: 12/362,906

(22) Filed: Jan. 30, 2009

(51) Int. Cl. A61B 1/267

(52) U.S. Cl. 600/190

(57) ABSTRACT

A medical device includes a laryngoscope having a handle extending along a handle direction; and a blade connected to the handle. The blade has an overall length along a longitudinal axis, an overall width along a lateral axis, and an overall height along a transverse axis, where the overall width is greater than the overall height, the overall length is greater than the overall width, and each of the axes is perpendicular to the other two axes. The blade includes a proximal region extending along a plane defined by the longitudinal axis and the transverse axis, and a distal region that is curved laterally relative to the longitudinal axis.

### TECHNICAL FIELD

This description relates to a laryngoscope blade.

### BACKGROUND

A laryngoscope is a medical instrument that is used to obtain a view of the vocal folds or cords and the glottis, which is the space between the cords. A rigid laryngoscope is utilized by anesthesia personnel for endotracheal intubation typically consists of a handle incorporating a power source such as batteries and an interchangeable blade with a bulb light source. Laryngoscopes used by otolaryngologists are found in many variations, used for various specialized tasks during endoscopy or surgery of the upper aerodigestive tract. The two main types of laryngoscope blades are the curved Macintosh blade and the straight Miller blade. The Macintosh blade sits anterior to the epiglottis and raises the epiglottis out of the visual pathway, while the Miller blade sits posterior to the epiglottis, trapping the epiglottis while exposing the glottis and vocal folds.

### SUMMARY

In one general aspect, a device includes a laryngoscope blade having an overall length along a longitudinal axis, an overall width along a lateral axis, and an overall height along a transverse axis, where the overall width is greater than the overall height and the overall length is greater than the overall width. Each of the axes is perpendicular to the other two axes. A proximal region of the blade extends along a plane defined the longitudinal axis and the transverse axis; and a distal region of the blade is curved laterally relative to the longitudinal axis.

Implementations may include one or more of the following features. For example, the proximal region can be substantially straight and can extend parallel with the longitudinal axis. Alternatively, the proximal region can be curved transversely relative to the longitudinal axis.

The distal region can be curved laterally at an angle between about 0° and about 60° relative to the longitudinal axis. The distal region can be curved laterally at an angle of about 45° relative to the longitudinal axis. The distal region can be curved laterally to the right of the left-right axis of the patient when fully inserted into a patient's oral cavity.

The distal region can be about three inches long.

The device can include a light source on the blade that is configured to provide illumination to the vocal cords when the blade of the laryngoscope is properly inserted into the patient.

The blade can have an arcuate cross section or a rectilinear cross section.

In another general aspect, a medical device includes a laryngoscope having a handle extending along a handle direction; and a blade connected to the handle. The blade includes a length along a longitudinal axis that is distinct from the handle direction, a width along a lateral axis, and a height along a transverse axis, where the width is greater than the height. The blade also includes a proximal region extending along a plane defined by the longitudinal axis and the transverse axis, and a distal region that is curved laterally relative to the longitudinal axis.

Implementations can include one or more of the following features. For example, the proximal region can be substantially straight and can extend parallel with the longitudinal axis. Alternatively, the proximal region can be curved transversely relative to the longitudinal axis.

The distal region can be curved laterally at an angle between 0°-60° relative to the longitudinal axis. The distal region can be curved laterally to the right of the left-right axis of the patient when fully inserted into a patient's oral cavity. The distal region can be about three inches long.

The device can include a light source on the blade that is configured to provide illumination to the vocal cords when the blade of the laryngoscope is properly inserted into the patient.

The blade can have an arcuate cross section or a rectilinear cross section.

In another general aspect, a distal region of a laryngoscope blade is inserted into an oral cavity of the patient so that the distal region is on a first side of a sagittal plane of the patient, a proximal region of the laryngoscope blade is on a second opposite side of the sagittal plane of the patient, and the proximal region extends parallel to the sagittal plane of the patient. The distal region is advanced far enough into the oral cavity to provide a view of the vocal cords such that the proximal region of the laryngoscope blade remains on the second opposite side of the sagittal plane of the patient.

Implementations can include one or more of the following features. For example, an endotracheal tube can be advanced through the oral cavity using the laryngoscope blade to enable a viewing of the vocal cords.

The distal region can be advanced into the oral cavity by advancing the distal region into the vallecula of the patient between the base of the tongue and the pharyngeal surface of the epiglottis. Alternatively, the distal region can be advanced into the oral cavity by advancing the distal region such that the distal region is posterior to the laryngeal surface and the epiglottis.

Other features and advantages will be apparent from the description, the drawings, and the claims.

### DESCRIPTION OF DRAWINGS

FIG. 1A is an anterior plan view of a laryngoscope blade;

FIG. 1B is a side view of the laryngoscope blade of FIG. 1A;

FIG. 1C is a posterior plan view of the laryngoscope blade of FIG. 1A;

FIG. 1D is a cross-sectional view of the laryngoscope blade of FIGS. 1A-C taken along line D-D shown in FIG. 1B;

FIG. 2A is a top view of a patient in which the laryngoscope blade of FIGS. 1A-D is partially inserted;

FIG. 2B is a top view of a patient in which the laryngoscope blade of FIGS. 1A-D is fully inserted;

FIG. 3A is a side cross-sectional view of a patient in which the laryngoscope blade of FIGS. 1A-D is partially inserted;

FIG. 3B is a side cross-sectional view of a close-up of the patient of FIG. 3A;

FIG. 3C is a side cross-sectional view of a patient in which the laryngoscope blade of FIGS. 1A-D is fully inserted;

FIG. 3D is a side cross-sectional view of a close-up of the patient of FIG. 3C;

FIG. 4 is a view of the larynx of the patient in FIGS. 2B and 3B as viewed by the clinician illustrating a comparative view of the vocal cordsthrough the laryngoscope blade of FIGS. 1A-D;

FIG. 5A is an anterior plan view of a laryngoscope blade;

FIG. 5B is a side view of the laryngoscope blade of FIG. 5A;

FIG. 5C is a posterior plan view of the laryngoscope blade of FIG. 5A;

FIG. 5D is a cross-sectional view of the laryngoscope blade of FIGS. 5A-C taken along line D-D shown in FIG. 5B;

FIG. 6A is a top view of a patient in which the laryngoscope blade of FIGS. 5A-D is partially inserted;

FIG. 6B is a top view of a patient in which the laryngoscope blade of FIGS. 5A-D is fully inserted;

FIG. 7A is a side cross-sectional view of a patient in which the laryngoscope blade of FIGS. 5A-D is partially inserted;

FIG. 7B is a side cross-sectional view of a close-up of the patient of FIG. 7A;

FIG. 7C is a side cross-sectional view of a patient in which the laryngoscope blade of FIGS. 5A-D is fully inserted;

FIG. 7D is a side cross-sectional view of a close-up of the patient of FIG. 7C;

FIG. 8 is a view of the larynx of the patient in FIGS. 6B and 7B as viewed by the clinician illustrating a comparative view of the vocal cords through the laryngoscope blade of FIGS. 5A-D; and

FIGS. 9A and 9B are side cross-sectional views of the patient in which the laryngoscope blade of, respectively, FIG. 3B and FIG. 7B, is fully inserted in the patient and an endotracheal tube is inserted.

### DETAILED DESCRIPTION

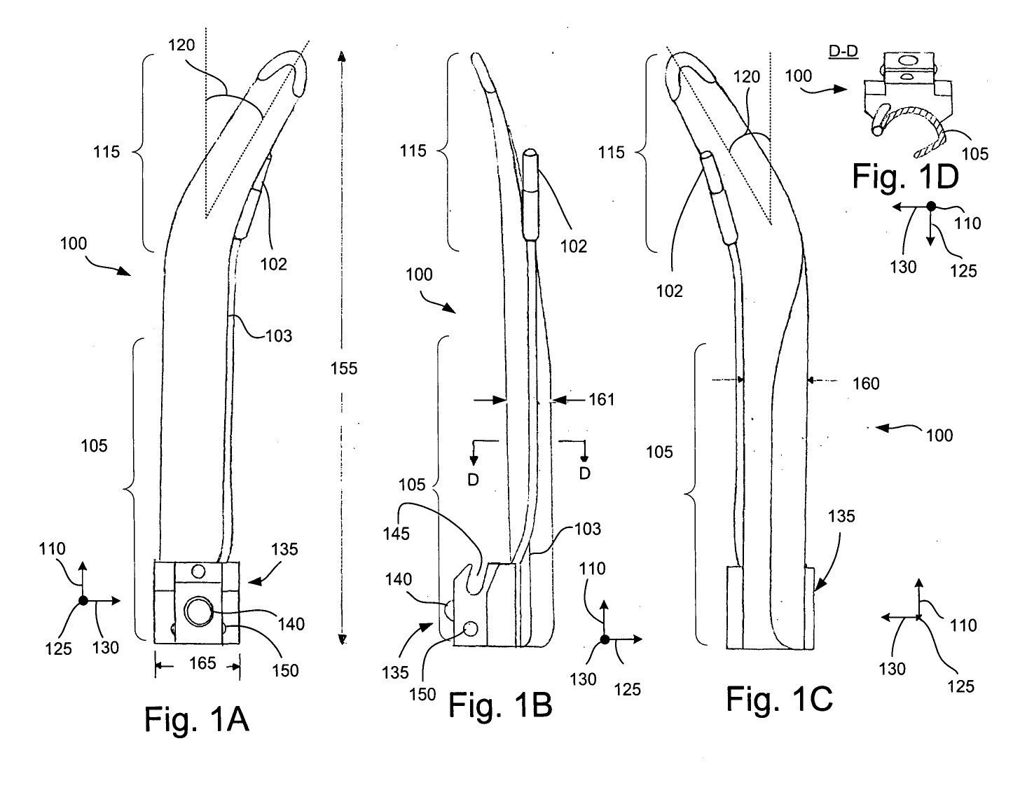

Referring to FIGS. 1A-1D, a laryngoscope blade 100 is designed to enable a clinician (for example, a physician, a nurse anesthetist, or an anesthesiologist) to more easily maneuver the blade 100 within a patient and to visualize the vocal cords of the patient. The patient can be a human patient or a non-human animal (in which case the clinician would be a veterinarian or a technician). The blade 100 is made of a material, for example, a metal such as stainless steel or a plastic, that is rigid enough to enable insertion into the oral cavity of the patient.

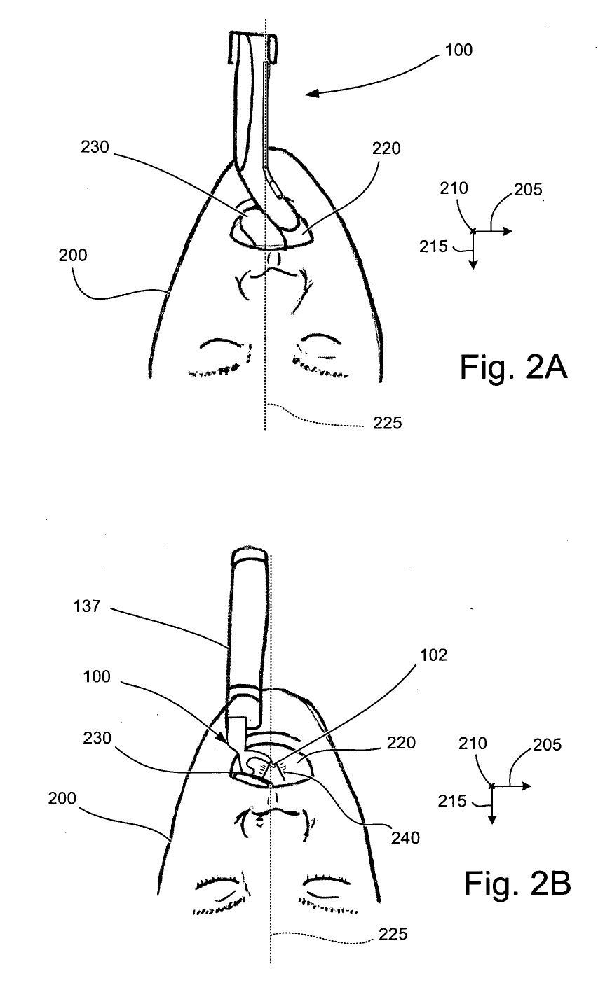

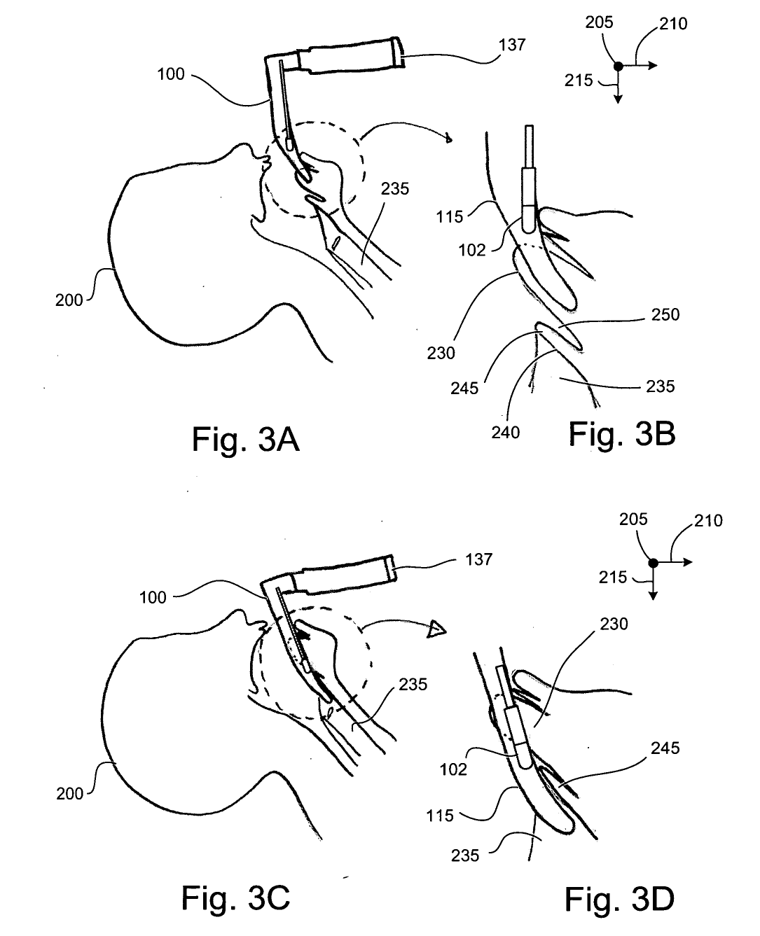

The blade 100 includes a proximal region 105 that extends along a plane defined by a longitudinal axis 110 and a transverse axis 125. When the blade 100 is inserted into the patient, the proximal region 105 is in the plane defined by the anteroposterior axis and the superior-inferior axis of the patient. Additionally, in this design, the proximal region 105 extends generally parallel with the longitudinal axis 110. The blade 100 also includes a distal region 115 that is curved at an angle 120 relative to the longitudinal axis 110 of the blade 100 about a transverse axis 125 that is perpendicular to the longitudinal axis 110 and to a left-right axis of the patient when the blade 100 is inserted into the patient. The distal region 115 of the blade 100 generally extends within the plane defined by the longitudinal axis 110 and a lateral axis 130 that is perpendicular to the longitudinal axis 110, though the distal region 115 can also be slightly curved along the transverse axis 125 relative to the longitudinal axis 110, as shown in FIG. 1B, by an angle of between 0-20°, for example. The lateral axis 130 is parallel with the left-right axis of the patient when the blade 100 is inserted into the patient. Moreover, the transverse axis 125 is in the plane defined by the anteroposterior axis and the superior-inferior axis of the patient when the blade 100 is inserted into the patient. For clarity, all human directional axes used herein are in reference to medical directional terms, and the human directional axes are shown in FIGS. 2A-3D.

The blade 100 also includes a light source 102 such as a halogen or a xenon bulb or a fiber optic delivery system. The light source 102 is positioned in the distal region 115 of the blade 100 and along an edge of the region 115 to provide sufficient light to enable the clinician to view the vocal cords when the blade 100 is inserted into the patient's oral cavity. Thus, the light source 102 can be positioned at the edge that is nearer to the vocal cords when the blade 100 is inserted into the patient's oral cavity. In the design shown herein, the light source 102 is positioned at a right edge 103 (relative to the patient's directional left-right axis) of the blade 100.

The angle 120 can be between about 0° and about 60°, and the value of the angle 120 is chosen to enable the clinician to more easily view the vocal cords of the patient after insertion of the blade 100 into the patient, as will be discussed and shown in greater detail below. For example, the angle 120 can be about 45° (for example, 45°±2°). As shown in FIG. 1D, the blade 100 has an arcuate cross section taken along the lateral axis 130.

The blade 100 includes an adapter 135 that is designed to connect both mechanically and electrically with a handle 137 (shown in FIGS. 2A-3D). The adapter 135 includes a contact 140 that makes electrical contact with a contact on the handle 137 when a hook 145 of the adapter 135 loops over a bar in an adapter (not shown) of the handle and one or more knobs 150 are slid into engagement with notches in the handle adapter (not shown). Additionally, the handle 137 includes a power supply such as a battery that supplies power to the light source 102 of the blade 100 when the contact 140 electrically connects with the handle contact.

The overall length 155 of the blade 100 taken along the longitudinal axis 110 can be about 6.125″, the width 160 of the blade 100 taken along the lateral axis 130 can be about 0.650″, and the overall width 165 of the adapter 135 taken along the lateral axis 130 can be about 0.875″ for an adult medium sized blade. The overall height 161 of the blade 100 taken along the transverse axis 125 can be about 0.5″. The width 165 is greater than the height 161. The length of the distal region 115 taken along the direction that is at the angle 120 relative to the longitudinal axis 110 can be about 3″.

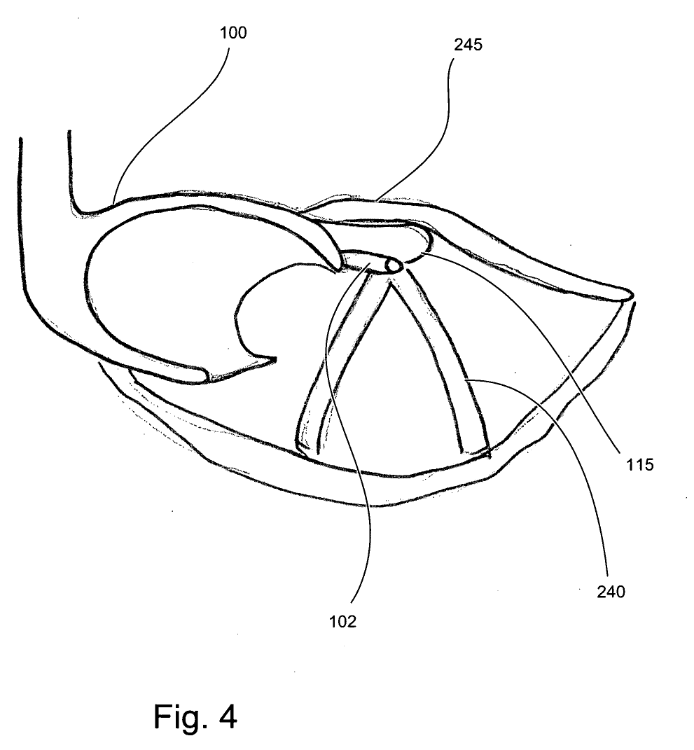

Referring also to FIGS. 2A-4, when the blade 100 is initially inserted into a patient 200, the lateral axis 130 is generally parallel with the left-right axis 205 of the patient 200, the transverse axis 125 is generally aligned with the superior-inferior axis 210 of the patient 200, and the longitudinal axis 110 is generally aligned with the anteroposterior axis 215 of the patient 200. The alignment of the blade 100 at all stages of the procedure is governed by the placement by the clinician; accordingly, the clinician's placement may not be exact due to human error, so that the alignment is really within a margin of human error. At this step in the procedure, the lateral axis 130 lies in the coronal plane, the transverse axis 125 is parallel with and off set from the sagittal plane, and the longitudinal axis 110 is parallel with and off set from the sagittal plane of the patient. When the blade 100 is fully inserted into the patient 200, the lateral axis 130 is generally parallel with the left-right axis 205 of the patient 200, the transverse axis 125 is along a plane that is defined by the superior-inferior axis 210 and the anteroposterior axis 215 of the patient 200, the longitudinal axis 110 is also along a plane that is defined by the superior-inferior axis 210 and the anteroposterior axis 215.

Initially, the clinician inserts distal region 115 of the blade 100 into the oral cavity 220 of the patient 200. As discussed above, the distal region 115 is curved at the angle 120 relative to the longitudinal axis 110 of the blade 100 about the transverse axis 125. The clinician maneuvers the blade 100 so that the distal region 115 is on the right side of a sagittal plane 225 of the patient and the proximal region 105 is on the left side of the sagittal plane 225 of the patient. The clinician is able to push the patient's tongue 230 to the left of the sagittal plane 225 as the distal region 115 is advanced through the oral cavity 220 because the proximal region 105 is generally on the left side of the plane 225 and the proximal region 105 serves to hold the tongue 230 to the left of the plane 225. In this way, as the clinician advances the distal region 115 far enough into the oral cavity 220 (as shown in FIGS. 2B, 3C, and 4) the clinician is provided with a view of the vocal cords 240 and also the opening into the patient's trachea 235 because the proximal region 105 remains generally on the left side of the sagittal plane 225 of the patient 200. Additionally, as the clinician advances the distal region 115 into the oral cavity 220, the distal region 115 is positioned beneath (that is, posterior to) the laryngeal surface 240 and the epiglottis 245 to push the epiglottis toward the base of the tongue 230 and block the vallecula 250.

Accordingly, by moving the proximal region 105 and the tongue 230 to the left side of the sagittal plane 225, the clinician obtains a significant increase in space near the plane 225 when compared with blades that lack a distal region 115 that is curved laterally relative to the longitudinal axis 110 to enable better visualization of the vocal cords 240 and provide better maneuverability of the endotracheal tube through the oral cavity 220 without interference from the tongue 230 or the blade 100.

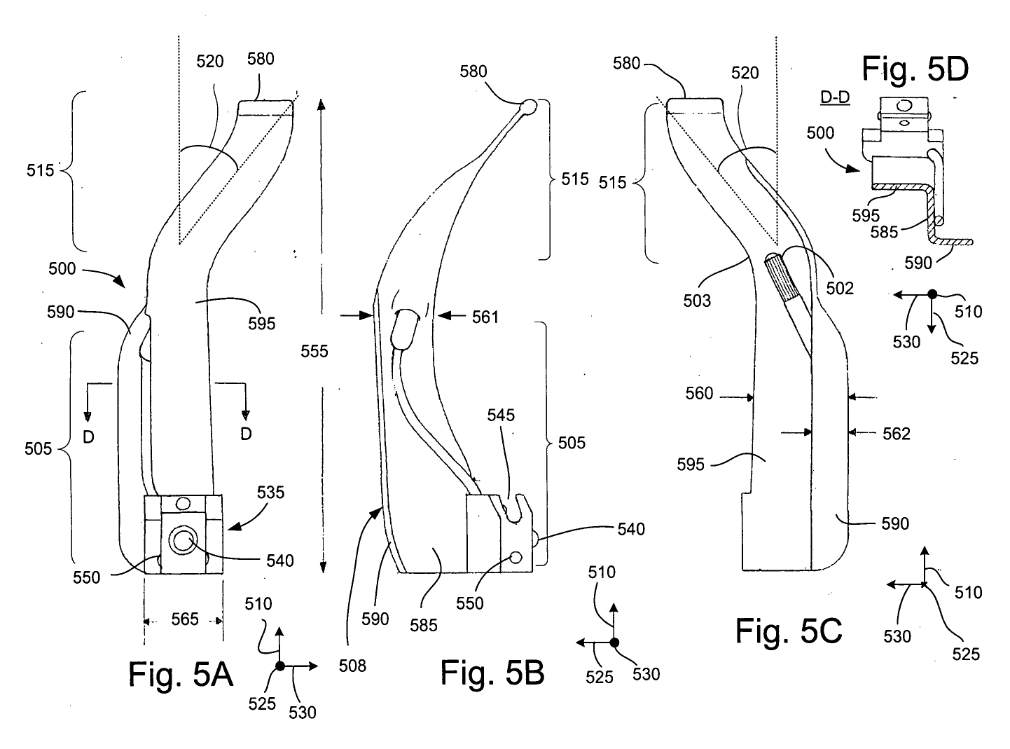

Referring to FIGS. 5A-5D, an alternative laryngoscope blade 500 is designed to enable a clinician (for example, a physician, a nurse anesthetist, or an anesthesiologist) to more easily maneuver the blade 500 within a patient and to visualize the vocal cords of the patient. The patient can be a human patient or a non-human animal (in which case the clinician would be a veterinarian or a technician). The blade 500 is made of a material, for example, a metal such as stainless steel or a plastic, that is rigid enough to enable insertion into the oral cavity of the patient.

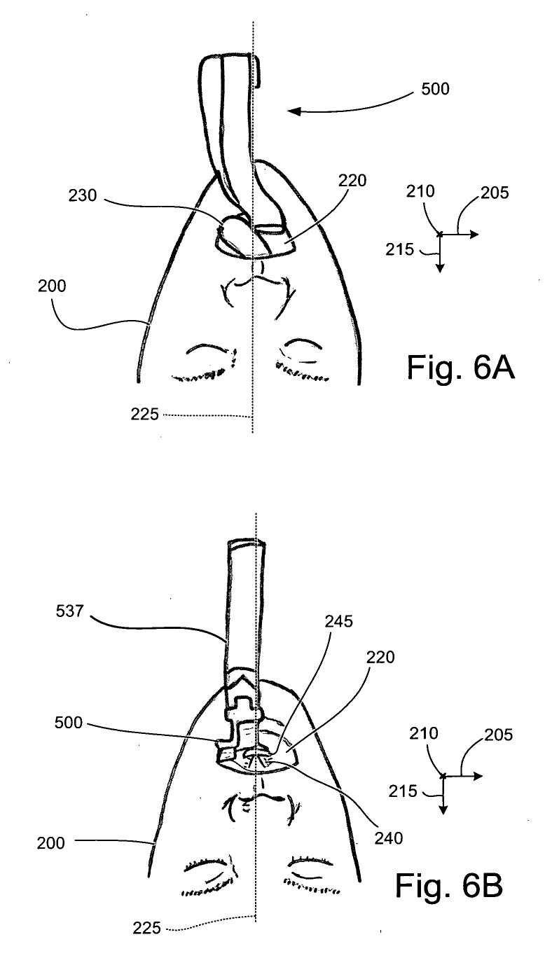

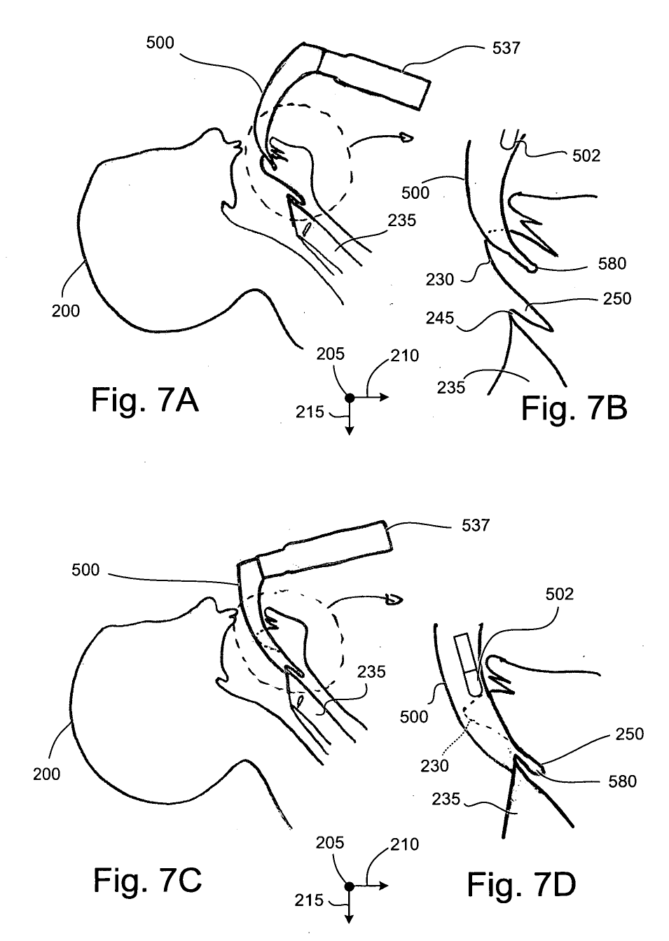

The blade 500 includes a proximal region 505 that extends along a plane defined by a longitudinal axis 510 and a transverse axis 525. When the blade 500 is inserted into the patient, the proximal region 505 is in the plane defined by the anteroposterior axis and the superior-inferior axis of the patient. The proximal region 505 can include at least a section 508 that is generally parallel with the longitudinal axis 510. The blade 500 also includes a distal region 515 that is curved at an angle 520 relative to the longitudinal axis 510 of the blade 500 about a transverse axis 525 that is perpendicular to the longitudinal axis 510 and to a left-right axis of the patient when the blade 500 is inserted into the patient. The distal region 515 and the proximal region 505 of the blade 100 can both be curved along the transverse axis 525 and about a lateral axis 530, as shown in FIG. 5B. The lateral axis 530 is perpendicular to the longitudinal axis 510 and is parallel with the left-right axis of the patient when the blade 500 is inserted into the patient. Moreover, the transverse axis 525 is in the plane defined by the anteroposterior axis and the superior-inferior axis of the patient when the blade 500 is inserted into the patient. For clarity, all human directional axes used herein are in reference to medical directional terms, and the human directional axes are shown in FIGS. 6A-7B.

The blade 500 also includes a light source 502 such as a halogen or a xenon bulb or a fiber optic delivery system. The light source 502 is positioned near the distal region 515 of the blade 500 to provide sufficient light to enable the clinician to view the vocal cords when the blade 500 is inserted into the patient's oral cavity. Thus, the light source 502 can be positioned at the edge that is nearer to the vocal cords when the blade 500 is inserted into the patient's oral cavity. In the design shown herein, the light source 502 is positioned to face a right edge 503 (relative to the patient's directional left-right axis) of the blade 500.

The angle 520 can be between about 0° and about 60°, and the value of the angle 520 is chosen to enable the clinician to more easily view the vocal cords of the patient after insertion of the blade 500 into the patient, as will be discussed and shown in greater detail below. For example, the angle 520 can be about 45° (for example, 45°±2°). As shown in FIG. 5D, the blade 500 has rectilinear cross section taken along the lateral axis 530. A rectilinear cross section means that the blade cross section has one or more straight lines that can mate with soft edges. The blade 500 includes a transverse planar portion 585 that extends between an upper lateral portion 590 and a lower lateral portion 595.

The blade 500 includes at a proximal end an adapter 535 that is designed to connect both mechanically and electrically with a handle 537 (shown in FIGS. 6A-7B). The adapter 535 includes a contact 540 that makes electrical contact with a contact on the handle 537 when a hook 545 of the adapter 535 loops over a bar in an adapter (not shown) of the handle 537 and one or more knobs 550 are slid into engagement with notches in the handle adapter (not shown). Additionally, the handle 537 includes a power supply such as a battery that supplies power to the light source 502 of the blade 500 when the contact 540 electrically connects with the handle contact. The blade 500 also includes a rounded distal tip 580 at a distal end of the distal region 515.

The overall length 555 of the blade 500 taken along the longitudinal axis 510 can be about 5.063″, the overall width 560 of the blade 500 taken along the lateral axis 530 can be about 1.00″, the width 562 of the upper lateral portion 590 of the blade 500 taken along the lateral axis 530 can be about 0.410″, and a width 565 of the adapter 535 taken along the lateral axis 530 (which is the sum of the widths of the upper lateral portion 590 and the lower lateral portion 595) can be about 0.875″ for an adult medium sized blade. The overall height 561 of the blade 500 can vary along the longitudinal axis 510 so that it is larger near the adapter 535 than near the distal tip 580. For example, the height 561 shown in FIG. 5B is about 0.625″. Nevertheless, the width 560 is greater than the height 561 at any location along the longitudinal axis 510. The length of the distal region 515 taken along the direction that is at the angle 520 relative to the longitudinal axis 510 can be about 3″.

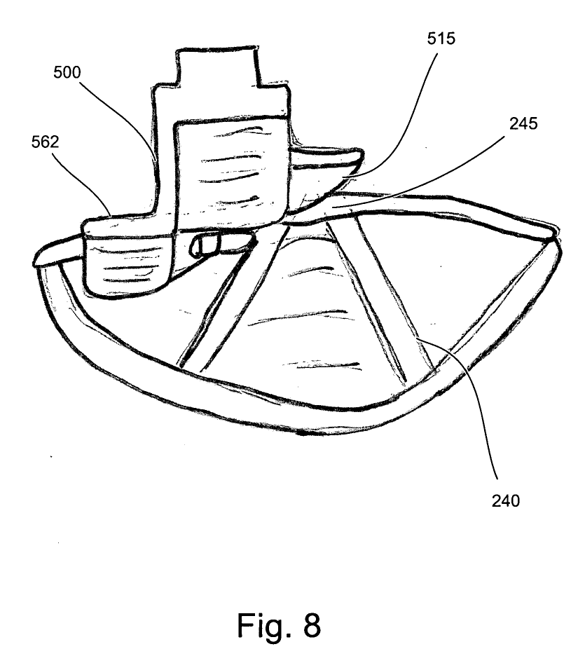

Referring also to FIGS. 6A-8, when the blade 500 is initially inserted into the patient 200, the lateral axis 530 is generally parallel with the left-right axis 205 of the patient 200, the transverse axis 525 is generally aligned with the superior-inferior axis 210 of the patient 200, and the longitudinal axis 510 is generally aligned with the anteroposterior axis 215 of the patient 200. As noted above, the alignment of the blade 500 is governed by the placement of the clinician and therefore it may be within an acceptable range as long as the blade 500 is placed in a manner that does not harm the patient but also enables the clinician to insert the blade 500 to view the patient's vocal cords. When the blade 500 is fully inserted into the patient 200, the lateral axis 530 is generally parallel with the left-right axis 205 of the patient 200, the transverse axis 525 is along a plane that is defined by the superior-inferior axis 210 and the anteroposterior axis 215 of the patient 200, the longitudinal axis 510 is also along a plane that is defined by the superior-inferior axis 210 and the anteroposterior axis 215.

Initially, the clinician inserts distal region 515 of the blade 500 into the oral cavity 220 of the patient 200. As discussed above, the distal region 515 is curved at the angle 520 relative to the longitudinal axis 510 of the blade 500 about the transverse axis 525. The clinician maneuvers the blade 500 so that the distal region 515 is on the right side of the sagittal plane 225 of the patient 200 and the proximal region 505 is on the left side of the sagittal plane 225 of the patient 200. The clinician is able to push the patient's tongue 230 to the left of the sagittal plane 225 as the distal region 515 is advanced through the oral cavity 220 because the proximal region 505 is generally on the left side of the plane 225 and the proximal region 505 serves to hold the tongue 230 to the left of the plane 225. In this way, as the clinician advances the distal region 515 far enough into the oral cavity 220 (as shown in FIGS. 6B, 7B, and 8) the clinician is provided with a view of the vocal cords 240 and also the opening into the patient's trachea 235 as the proximal region 505 remains generally on the left side of the sagittal plane 225 of the patient 200. Additionally, as the clinician advances the distal region 515 into the oral cavity 220, the distal tip 580 is positioned above (that is, anterior to) the epiglottis 245 and rests inside the vallecula 250.

Accordingly, by moving the proximal region 505 and the tongue 230 to the left side of the sagittal plane 225, the clinician obtains a significant increase in space near the plane 225 when compared with blades that lack a distal region 515 that is curved laterally relative to the longitudinal axis 510 to enable better visualization of the vocal cords 240 and provide better maneuverability of the endotracheal tube through the oral cavity 220 without interference from the tongue 230 or the blade 500.

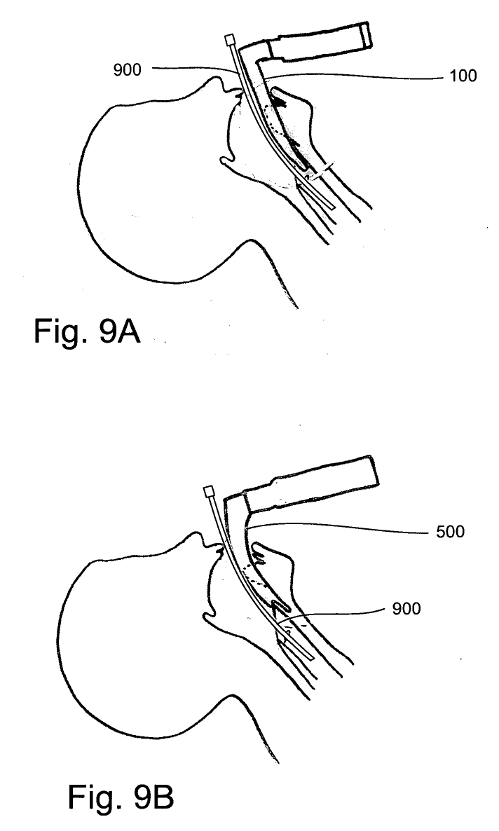

Referring also to FIGS. 9A and 9B, in both cases, the clinician can use the laryngoscope blade 100 or 500 to maneuver an endotracheal tube 900 after the blade 100, 500 has been properly inserted into the patient's oral cavity 220. In this case, the clinician advances the endotracheal tube 900 through the oral cavity 220, along and past the laryngoscope blade 100, 500, through the vocal cords 240, and into the trachea 235.

Other implementations are within the scope of the following claims. For example, the laryngoscope blade can be made of any material (such as plastic or metal) that is suitable for the particular application in which the blade will be used, for example, to expose the vocal cords 240.

In the blades 100, 500 shown above, the angles 120, 520 are toward the right side of the sagittal plane 225 of the patient 200. However, the blades 100, 500 could be designed as mirror images about the sagittal plane 225 of the patient 200 as the blades shown herein and in such mirror imaged designs, the angles 120, 520 would be toward the left side of the sagittal plane 225 of the patient 200. Such a mirror imaged design might be useful for clinicians who are left handed.

The blades 100, 500 can have cross sections other than the arcuate and rectilinear cross sections shown above.

The laryngoscope blade can have a length and a width that is suitable for the particular application of the blade. For example, a blade to be used on an infant can have a smaller size than the sizes given above in reference to the blades 100, 500, a blade to be used on a large adult can have a larger size than the sizes given above in reference to the blades 100, 500. For example, laryngoscope blades such as Miller and Macintosh blades come in a range of sizes that are suitable for use on a premature baby, an infant, a child, an average adult, and a large adult, respectively.

What is claimed is:

1. A device comprising:
a laryngoscope blade having an overall length along a longitudinal axis, an overall width along a lateral axis, and an overall height along a transverse axis, where the overall width is greater than the overall height, the overall length is greater than the overall width, and each of the axes is perpendicular to the other two axes;
a proximal region of the blade extending along a plane defined by the longitudinal axis and the transverse axis; and
a distal region of the blade curved laterally relative to the longitudinal axis.

2. The device of claim 1, wherein the proximal region is substantially straight and extends parallel with the longitudinal axis.

3. The device of claim 1, wherein the proximal region is curved transversely relative to the longitudinal axis.

4. The device of claim 1, wherein the distal region is curved laterally at an angle between about 0° and about 60° relative to the longitudinal axis.

5. The device of claim 1, wherein the distal region is curved laterally to the right of the left-right axis of the patient when fully inserted into a patient's oral cavity.

6. The device of claim 1, wherein the distal region is about three inches long.

7. The device of claim 1, further comprising a light source on the blade and configured to provide illumination to the vocal cords when the blade of the laryngoscope is properly inserted into the patient.

8. The device of claim 1, wherein the blade has an arcuate cross section.

9. The device of claim 1, wherein the blade has a rectilinear cross section.

10. The device of claim 1, wherein the distal region is curved laterally at an angle of about 45° relative to the longitudinal axis.

11. A medical device, comprising:
a laryngoscope comprising:
a handle; and
a blade connected to the handle, the blade having an overall length along a longitudinal axis, an overall width along a lateral axis, and an overall height along a transverse axis, where the overall width is greater than the overall height, the overall length is greater than the overall width, and each of the axes is perpendicular to the other two axes, wherein the blade includes a proximal region extending along a plane defined by the longitudinal axis and the transverse axis, and a distal region that is curved laterally relative to the longitudinal axis.

12. The device of claim 11, wherein the proximal region is substantially straight and extends parallel with the longitudinal axis.

13. The device of claim 11, wherein the proximal region is curved transversely relative to the longitudinal axis.

14. The device of claim 11, wherein the distal region is curved laterally at an angle between 0°-60° relative to the longitudinal axis.

15. The device of claim 11, wherein the distal region is curved laterally to the right of the left-right axis of the patient when fully inserted into a patient's oral cavity.

16. The device of claim 11, wherein the distal region is about three inches long.

17. The device of claim 11, further comprising a light source on the blade and configured to provide illumination to the vocal cords when the blade of the laryngoscope is properly inserted into the patient.

18. The device of claim 11, wherein the blade has an arcuate cross section.

19. The device of claim 11, wherein the blade has a rectilinear cross section.

20. A method comprising:
inserting a distal region of a laryngoscope blade into an oral cavity of the patient so that the distal region is on a first side of a sagittal plane of the patient, a proximal region of the laryngoscope blade is on a second opposite side of the sagittal plane of the patient, and the proximal region extends parallel to the sagittal plane of the patient; and
advancing the distal region far enough into the oral cavity to provide a view of the vocal cords such that the proximal region of the laryngoscope blade remains on the second opposite side of the sagittal plane of the patient.

21. The method of claim 20, further comprising:
advancing an endotracheal tube through the oral cavity using the laryngoscope blade to enable a viewing of the vocal cords.

22. The method of claim 20, wherein advancing the distal region into the oral cavity includes advancing the distal region into the vallecula of the patient between the base of the tongue and the pharyngeal surface of the epiglottis.

23. The method of claim 20, wherein advancing the distal region into the oral cavity includes advancing the distal region such that the distal region is posterior to the laryngeal surface and the epiglottis.
